(19) United States
(12) Patent Application Publication
(10) Pub. No.: US 2014/0030751 A1
(43) Pub. Date: Jan. 30, 2014

(54) Devices and Methods for the Fast Photochemical Oxidation of Proteins

(71) Applicant: University of Georgia Research Foundation, Inc., Athens, GA (US)

(72) Inventor: Joshua S. Sharp, Bogart, GA (US)

(21) Appl. No.: 13/951,708

(22) Filed: Jul. 26, 2013

Related U.S. Application Data
(60) Provisional application No. 61/676,363, filed on Jul. 27, 2012.

(51) Int. Cl. G01N 33/68

(52) U.S. Cl. CPC G01N 33/6803
USPC 435/23; 422/67; 436/43; 435/288.7

(57) ABSTRACT

Embodiments of the present disclosure provide for devices and methods that can be used in a process for the fast photochemical oxidation of proteins, and the like.

### CLAIM OF PRIORITY TO RELATED APPLICATION

This application claims priority to co-pending U.S. provisional application entitled “DEVICES AND METHODS FOR THE FLASH PHOTOCHEMICAL OXIDATION OF PROTEINS” having Ser. No. 61/676,363, filed on Jul. 27, 2012, which is entirely incorporated herein by reference.

### FEDERAL SPONSORSHIP

This invention was made with Government support under Contract/Grant No. 1R01GM096049-01A1, 5P41RR005351-23 and 8P41GM103390-23, awarded by the National Institute of Health. The Government has certain rights in this invention.

### BACKGROUND

Hydroxyl radical protein footprinting coupled with mass spectrometry is a powerful tool used to probe protein structure, interactions, and conformational changes in the solution phase. A hydroxyl radical dosimeter is used to ensure that different proteins oxidized under the same amount of radical. However, AlexaFluor™ is not an ideal dosimeter for hydroxyl radicals: it is a loss-of-signal dosimeter which lowers its sensitivity, and has several oxidation products, each with different properties. Thus, there is a need to overcome these deficiencies.

### SUMMARY

Embodiments of the present disclosure provide for devices and methods that could be used in a process for the fast photochemical oxidation of proteins and the like.

In an embodiment, the automated device, includes: a sample handling system for transporting at least a sample, a photoactive reagent mixture, and a sample mixture; a light source system for initiating a reaction in the sample mixture; a sample flow and control system for transporting at least the sample, the photoactive reagent mixture, and the sample mixture; a photometer system to measure light source output; and an analysis system for analyzing the sample mixture, where the analysis system measures the amount of radical generated in the sample mixture.

In an embodiment, the automated method, includes: mixing a sample with a photoactive reagent mixture to form a sample mixture; exposing the sample mixture to light energy to initiate activation of the reaction in the sample mixture; mixing the sample mixture with one or more other reagents; measuring the amount of radicals in the sample mixture; and analyzing the sample mixture.

### BRIEF DESCRIPTION OF THE DRAWINGS

Many aspects of this disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1.1 illustrates an embodiment of the device.

FIG. 1.2 illustrates another embodiment of the device.

FIGS. 2.1A and 2.2 illustrate the direct infusion MS analysis of intact ubiquitin with glutamine amide as a scavenger. FIG. 2.1 illustrates an intact mass spectrum of 8 μM ubiquitin with 20 mM glutamine in 50 mM ammonium bicarbonate buffer after irradiation, while FIG. 2.2 is mass spectrum of specific areas of the mass spectra in FIG. 2.1A.

FIGS. 2.1B and 2.3 to 2.4 illustrate the direct infusion MS analysis of intact ubiquitin with tyrosine amide as a scavenger. FIG. 2.B1 illustrates an intact mass spectrum of 8 μM ubiquitin with 5.4 mM tyrosine amide in 50 mM ammonium bicarbonate buffer after irradiation. FIGS. 2.3 and 2.4 illustrate specific areas of the mass spectra in FIG. 2.1B

FIG. 2.5 illustrates Table 1.

FIG. 2.6 illustrates Table 2.

FIG. 2.7A and 2.7B illustrate the oxidization percentage of tyrosine amide with ubiqutin in tris-HCl buffer. FIG. 2.7A illustrates a graph that shows 5.4 mM tyrosine amide with 8 μM ubiqutin in different concentrations of Tris buffer (1 mM to 100 mM) irradiated with 100 mM H₂O₂. FIG. 2.7B illustrates a graph that shows 5.4 mM tyrosine amide with 8 μM ubiqutinin in 50 mM Tris buffer irradiated with different concentrations of H₂O₂ (1 mM to 100 mM).

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### General Discussion

Embodiments of the present disclosure provide for devices and methods that can be used in a process for the fast photochemical oxidation of proteins, and the like. Embodiments of the present disclosure enable automation of the process known as Fast Photochemical Oxidation of Proteins (FPOP). FPOP is a method through which a protein is mixed with a photoreactive compound (e.g. hydrogen peroxide) and scavenging solutions, flashed with a very brief pulse of light of the appropriate wavelength to activate the photoreactive compound (e.g. a pulse from an excimer laser), and then longer-lived reaction intermediates are quenched with a quench solution (e.g., catalase and methionine amide). Modified proteins are then digested with trypsin and injected into an analysis system (e.g., an LC-MS system) in the integrated automated system.

FPOP can be used to compare the structures of protein pharmaceuticals, which is an area of intense interest and importance for the development of biosimilars and the validation of protein structure and stability during pharmaceutical process and formulation development. However, FPOP can not be practically used (e.g., reproducibility) since there are no systems that can automate FPOP and provide a feedback loop to manage the reaction, specifically regulating the amount of available radicals. Embodiments of the present disclosure provide for the automation of the entire process of FPOP and the subsequent analysis of the product by LC-MS.

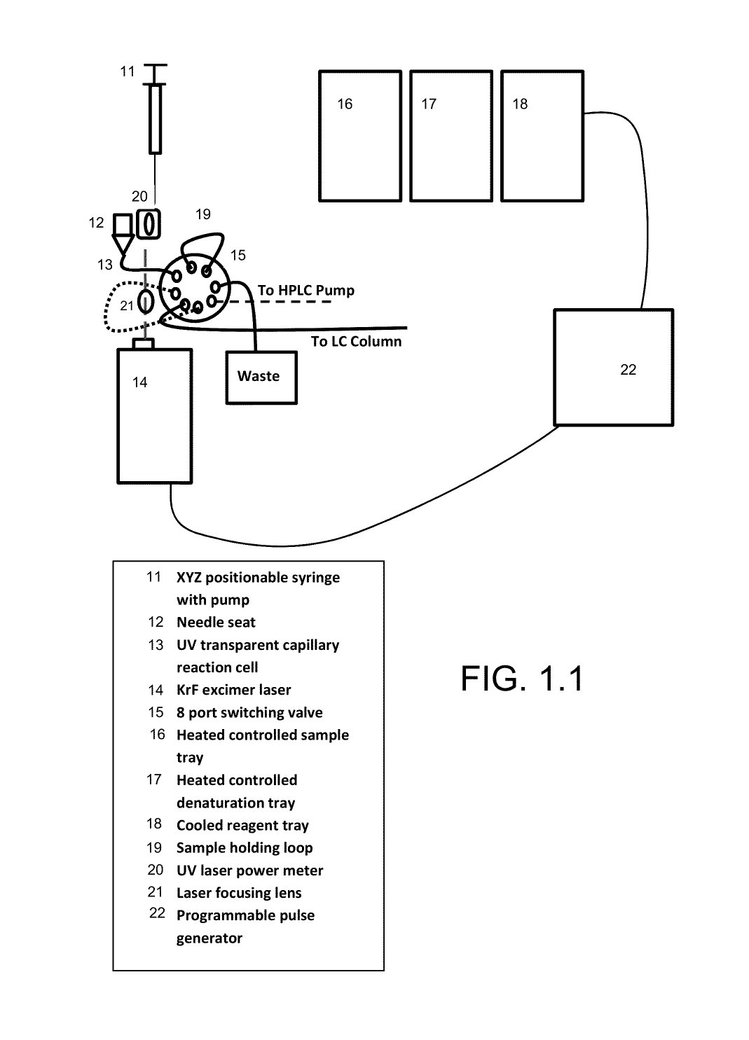

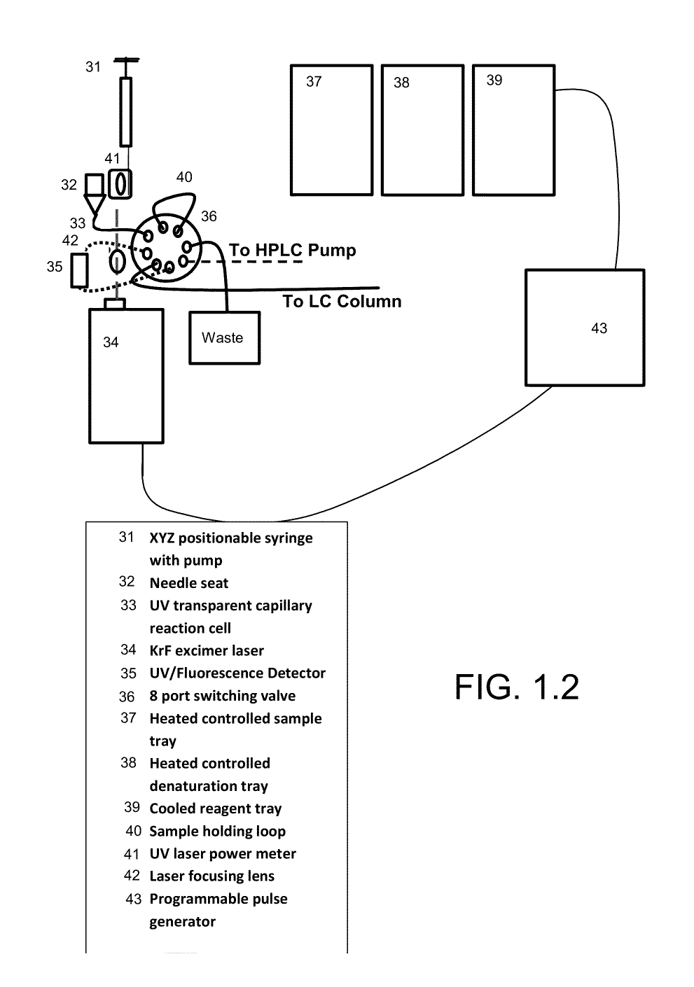

An embodiment of the device includes: a sample handling system; a focused pulsed light source system (e.g., a laser); a sample flow and control system; a photometer system to measure light source output; an analysis system (e.g., LC-MS system) that can be used for measuring the amount of radical generated during the FPOP reaction and used to analyze the modified proteins after the FPOP reaction is complete, where a feedback loop can be used to adjust one or more parameters to optimize the reaction. An embodiment of an exemplary device is shown FIG. 1.1, and additional details are provided in Example 1.

Another embodiment of the device includes: a sample handling system; a focused pulsed light source system (e.g., a laser); a sample flow and control system; a photometer system to measure light source output; a UV/fluorescence detector system for measuring the amount of radical generated during the FPOP reaction; and an analysis system used to analyze the modified proteins after the FPOP reaction is complete, where a feedback loop can be used to adjust one or more parameters to optimize the reaction. An embodiment of an exemplary device is shown FIG. 1.2, and additional details are provided in Example 1.

Now having described the devices and methods in general, the following describes additional details regarding the various embodiments and the components of these embodiments.

In an embodiment a sample handling system and/or the sample flow and control system can be used to handle and/or transport (e.g., flow, mix, and the like) the sample, sample mixture, and reagents throughout the device. In an embodiment these two systems can operate to accomplish transporting the sample and reagents separately or in combination. In an embodiment, the sample handling system can include a robotic liquid handle, and/or one or more x-, y-, and/or z-positionable syringes or fluid transfer devices to introduce the sample mixture to an area to be exposed to a light source and subsequently moved to an area for further reaction before being introduced to the analysis system. In an embodiment, the sample handling system and/or the sample flow and control system can include a multi-position valve assembly. In addition, the sample handling system and/or the sample flow and control system can be used to control (e.g., measure and control the rate of sample flow) the flow of the fluid through the flow path and elsewhere in the device. In an embodiment, one or both of these systems can include a temperature-control system to control the temperature at one or more areas of the device. Pumps, syringes, flow meters, flow controllers, and appropriate flow paths (e.g., tubing) can be used within the device to transport, hold, and/or mix the sample, sample mixture, and reagents from the initial mixing of the components, photoinitiation reaction using the light source to initiate oxidation of the proteins, flow to or through the UV/fluorescence detector system or mass spectrometry system for measuring the amount of radical generated, reaction of the proteins with proteases, and analysis of the modified proteins using the analysis system.

In an embodiment, the sample handling system and/or the sample flow and control system can include mixing the sample with a photoactive reagent mixture in a reaction cell to produce a sample mixture. In an embodiment, the photoactive reagent mixture can include a photoactive agent and a radical scavenger, and a radical dosimeter (e.g., depending on the system a UV/fluorescence-based radical dosimeter (e.g., hydrogen peroxide from about 5 to 50 mM, glutamine scavenger from about 7 to 67 mM, and AlexaFluor from about 5 μM to 20 μM) or a mass-based radical dosimeter (e.g., tyrosine amide)(used in the mass spectrometry system embodiment)). It should be noted that the ratios of the components can depend upon the quantum yield for the photoactive agent, the reactivity for the activated agent to the scavenger, the expected peak concentration of activated photoactive reagent, and the like.

In an embodiment, the photoactive reagent mixture can include a mixture of hydrogen peroxide, glutamine radical scavenger, and AlexaFluor™ dosimeter reagent. In an embodiment, the photoactive reagent mixture can include a mixture of hydrogen peroxide, a radical scavenger (e.g., glutamine amide), and a mass-based radical dosimeter (e.g., tyrosine amide). In an embodiment, other photoactive agents can include: persulfate, iodobenzoic acid, aryl azides, and benzophenone. In an embodiment, other radical scavengers can include tyrosine amide, tris, histidine amide, and methionine amide. In an embodiment, the tyrosine amide can be used as the radical scavenger and the radical dosimeter. In an embodiment, another UV/fluorescent dosimeter can include terephthalic acid.

In an embodiment, the mass-based radical dosimeter can include compounds that include one more of the following characteristics: moderately to highly reactive with hydroxyl radicals, soluble in water at micromolar to millimolar concentrations, poorly reactive with the photoreagent (e.g., hydrogen peroxide), a single isobaric set of major reaction products with hydroxyl radicals, moderate to poor absorbance at 247 nm, and ionizes by electrospray. In an embodiment, the mass-based radical dosimeter can include tyrosine amide. In an embodiment, the mass-based radical dosimeter can include a hydrophilic substituted phenyl compound with at least one protonation site. In an embodiment, the hydrophilic substituted phenyl compound can include tyrosine, tryptophan, and phenylalanine, and aromatic compounds like acetaminophen and substituted anilines, and the like.

In an embodiment, the sample handling system and/or the sample flow and control system can be used to introduce a quenching reagent (e.g., catalase, methionine amide, and the like) to quench longer lived reaction products. In addition, the sample handling system can be used to introduce a denaturation solution (e.g., containing 6 M guanidinium HCl and 10 mM DTT) to the sample mixture. Furthermore, the sample handling system and/or the sample flow and control system can introduce a dilute buffered solution of trypsin to the sample mixture. The sample handling system and/or the sample flow and control system can be used to transport and mix the various reagents and the sample mixture, while also adjusting the temperature as needed for the various reactions. Additional details are provided in Examples 1 and 2.

In an embodiment, transportation of the sample mixture can include moving (e.g., pushing) the sample mixture through a pulsed light-transparent window within the focused pulsed light beam path. The light energy can initiate a reaction by activating the photoactive agent to start the fast photochemical oxidation of proteins in the sample mixture.

In an embodiment, the light source system includes a focused pulsed light source system such an excimer laser or other light source of appropriate power, wavelength, and the like, that can activate the photoactive agent. In an embodiment, the device includes an area (or portion of a capillary tube) where the sample can be flowed, where the area includes a window transparent to the wavelength of the light source so that the light passes through the window and initiates the photoactive agent. In an embodiment, at an appropriate time the sample mixture can be mixed with the photoactive reagent mixture and then introduced to the chamber including the transparent window. In an embodiment, the light source system includes a light source pulse timer to control the exposure of each volume of the sample mixture to pulses of light. In an embodiment, the timing pulse can be designed so that the sample mixture is activated to the desired level and can be controlled using a programmable control module as mentioned below. In addition, the light source system can include a mirror(s), a focusing lens(s), and other appropriate equipment for focusing and directing a light source.

In an embodiment, the light source system includes a photometer system to measure the output from the light source. In an embodiment, the photometer system includes a photometer that is adjacent or in-line with the light source. In an embodiment, the photometer system can be interfaced with one or more systems (e.g., light source system) of the device. In an embodiment, the signal measured by the photometer system can be used in a feedback loop (e.g., a programmable control module) to the light source system so that that light energy can be modified as needed. In an embodiment, the photometer system can include a mirror(s), a focusing lens(s), and other appropriate equipment for focusing and directing the light to the photometer.

Once the sample mixture has been irradiated with the light source and further treated (e.g., quenched, denatured, treated with trypsin, and the like), the sample mixture can be introduced to a system to measure the amount of available radical generated. As described herein, one or more parameters can be adjusted based on the available radicals to ensure the reaction proceeds as desired. As mentioned above, a mass spectrometry system (i.e., the same one used in the analysis system or a separate mass spectrometry system) and/or a UV/fluorescence detector system can be used to measure the amount of available radicals generated. In an embodiment, the sample mixture is flowed through a tube to the mass spectrometry system (e.g., LC-MS system).

The radical dosimeter, whether it is a mass-based radical dosimeter (e.g., tyrosine amide) or a fluorescent agent) measures the amount of available radical, taking into account the amount of radical generated and the amount consumed by the sample, the buffer, and the like. In an embodiment where the radical dosimeter is a mass-based radical dosimeter such as tyrosine amide, the amount of available radical can be measured by virtue of a reaction at a stable, well-known rate that varies by the concentration of available hydroxyl radical and the concentration of tyrosine amide. The reaction of tyrosine amide with hydroxyl radical leads almost exclusively to the net addition of one oxygen atom, which can be identified and quantified easily by mass spectrometry. Additional details are provided in Example 2.

In an embodiment, the UV/fluorescence detector system can be used for measuring the amount of radical generated at one or more positions within the device. In an embodiment, the sample mixture is flowed through a tube that passes through or adjacent a UV/fluorescence detector. In an embodiment, the UV/fluorescence detector system can include a mirror(s), a focusing lens(s), and other appropriate equipment for detecting and measuring UV and/or fluorescent energy.

In an embodiment, the device includes one or more programmable control modules to develop and/or control reaction sequences of the sample so as to automate FPOP and additional analysis. As mentioned above, a programmable control module can be used in a feedback loop for the light source system and the photometer system as the light energy is pulsed through the sample mixture. In an embodiment, part of the feedback loop includes the amount of radical present in the sample as measured, so that the light energy can be adjusted up or down as needed. In an embodiment, one or more control modules can be used to control the automatic selection of the sample, mixing of the sample with the photoactive reagent mixture and other reagents, introduction of the sample mixture to the chamber to be exposed to light energy and the like. As mentioned above, a programmable control module can be used in a feedback loop for the light source system and the photometer system as the light energy is pulsed through the sample mixture. One or more other programmable control modules can be used to control the automation of one or more of the systems, control communication and adjustment of parameters between or among one or more of the systems, and the like.

In an embodiment, the analysis system can be used to analyze the sample before and/or after reactions conducted on the sample mixture and be integrated into the feedback loop to modify the reaction as needed. In an embodiment, the sample mixture can be analyzed after the treatment (e.g., quenched, denatured, treated with trypsin, and the like) using a LC-MS system to detect the proteins and/or protein fragments. In another embodiment, the analysis system can include another type of analytical instrument.

In another embodiment, the device includes: a robotic liquid handler for mixing sample with the photoreactive compound, radical dosimeter agent, a scavenger, and/or a quench solution; a focused pulsed light source (e.g., excimer laser); a sample flow path to push the sample through a pulsed light-transparent window within the focused pulsed light beam path; a flow controller to measure and control the rate of sample flow through the flow path; a light source pulse timer to control the number of pulses of light each volume of the sample is exposed to; a photometer to measure light source output; a temperature-controlled sample array; a switching valve to divert liquid handling; a programmable control module to develop reaction sequences; optionally, a UV/fluorescence detector for measuring the amount of radical generated or use of the analysis system to measure the amount of radical generated; and an analysis system such as a LC-MS system, where one or more control modules can adjust one or more parameters to control/modify the reaction in real time or near real time.

Additional details regarding the device and how sample and sample mixture are moved around and through the device are described in reference to FIGS. 1.1 and 1.2, but the sample and sample mixture flow mentioned in FIGS. 1.1 and 1.2 can be also applied to other embodiments of the present disclosure described herein.

It should be noted that other devices do not include the combination of the focused laser light source, reaction cell, and/or the syncing of the laser source system with the fluid handling/autosampler equipment, and they also do not use the in-line pre-LC UV/fluorescence detector for monitoring radical dose.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

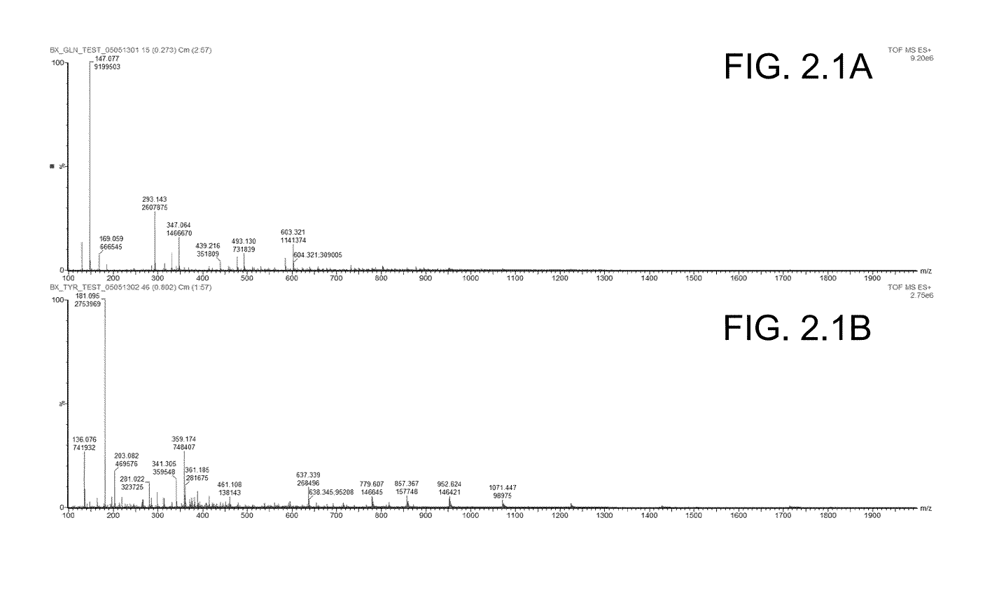

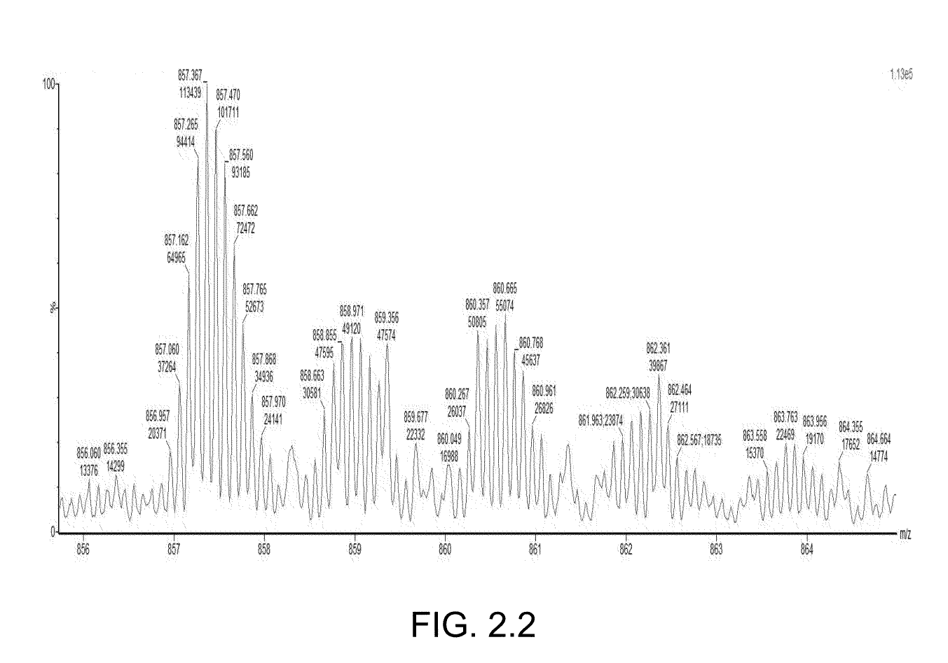

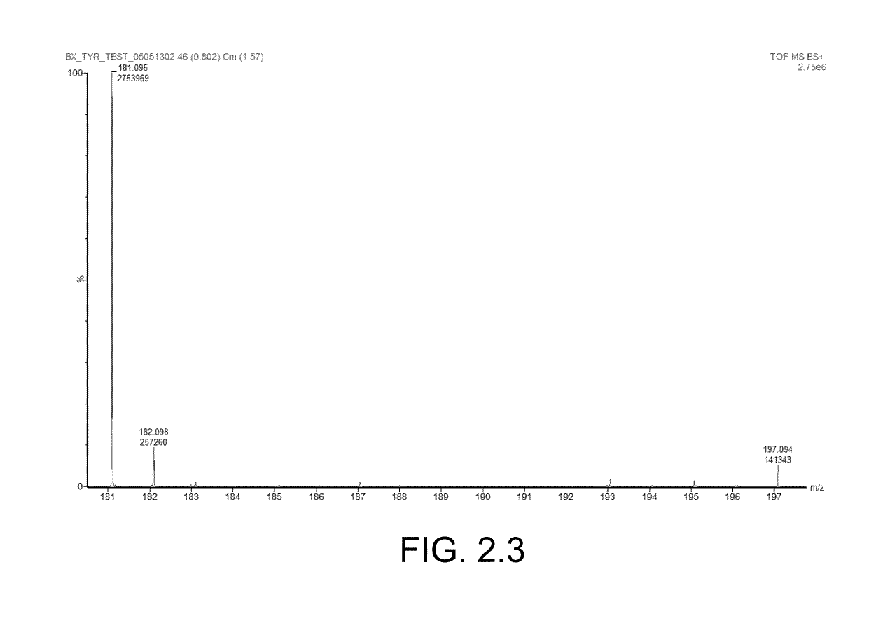

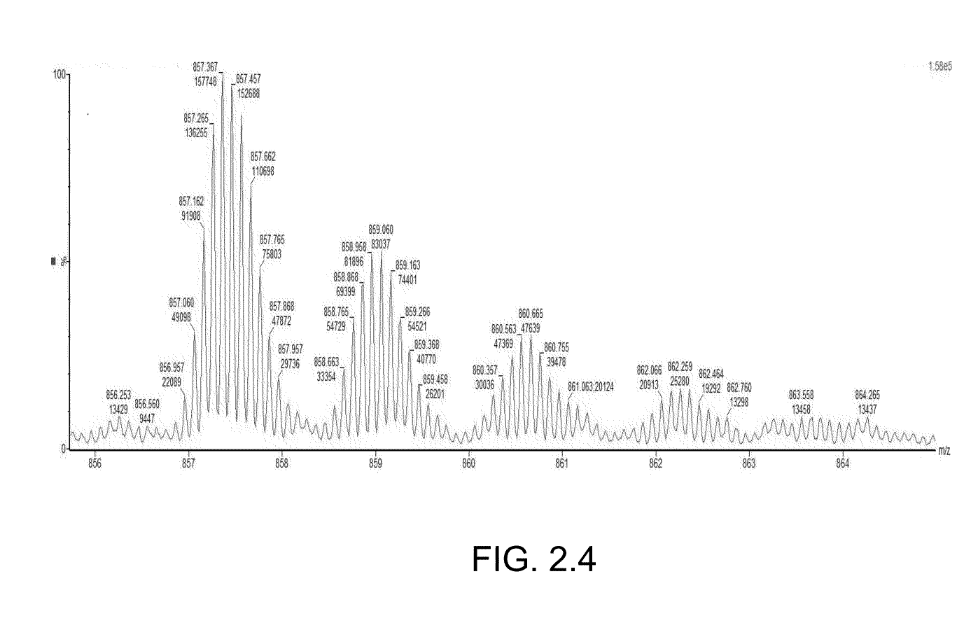

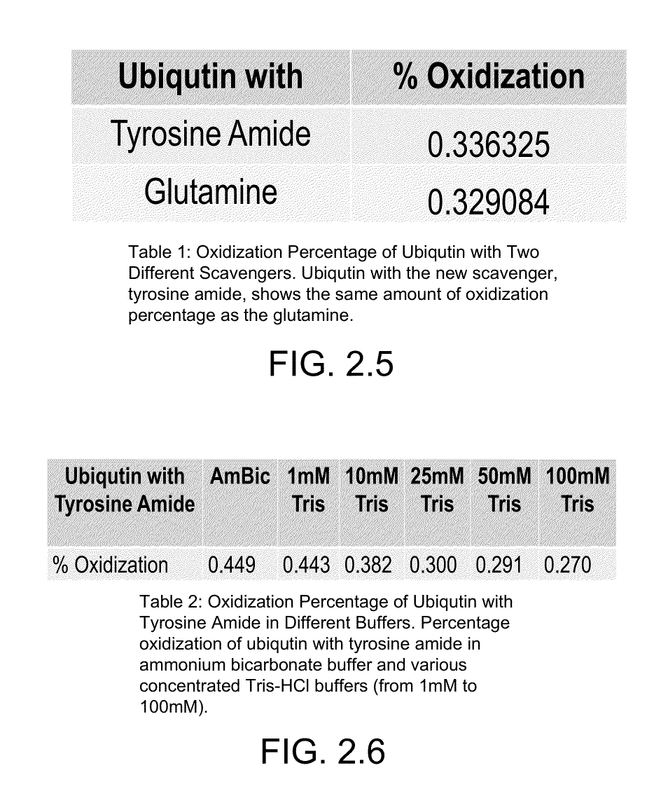

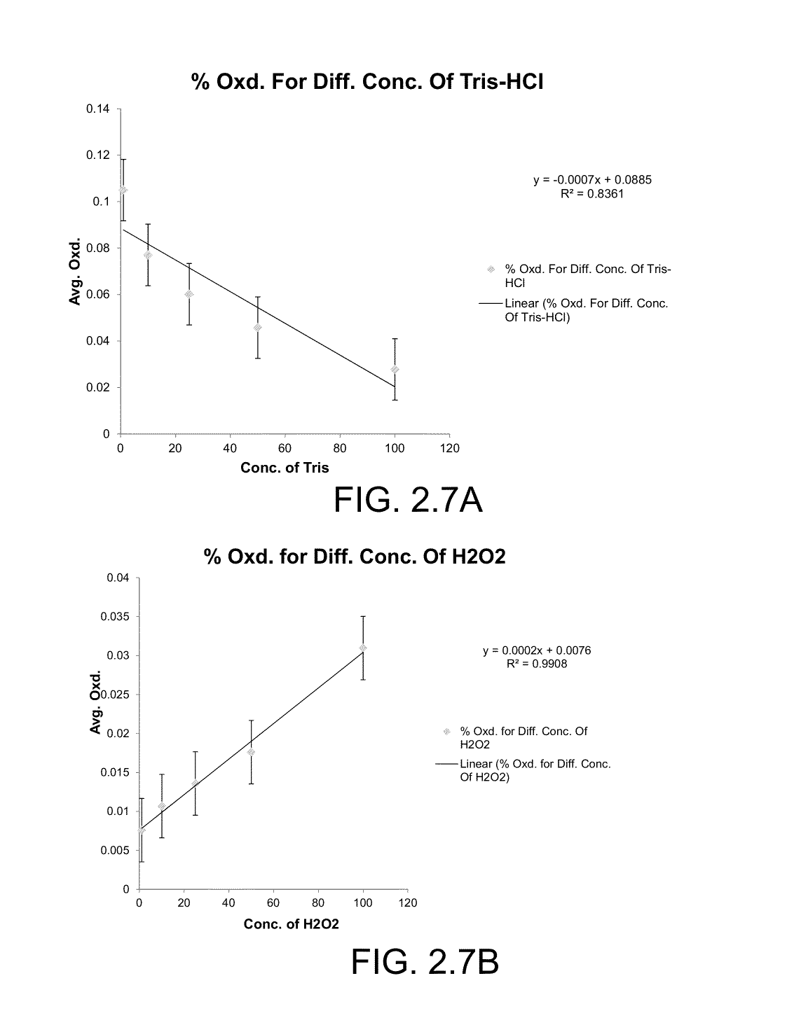

Embodiments of the present disclosure include a device that is designed to automate the process of Fast Photochemical Oxidation of Proteins (FPOP) using a mass spectrometry of the analysis system for measuring the amount of radical generated in the sample mixture. A diagram of the device is given in FIG. 2.1. The sample can be incubated in the heated sample tray (17) at a preset temperature (e.g., 37° C.) until ready for oxidation. The XYZ positionable syringe with pump (11) will deposit the photoactive reagent mixture (e.g., a mixture of hydrogen peroxide, a radical scavenger (e.g., glutamine amide or tyrosine amide), and radical dosimeter reagent (e.g., tyrosine amide)) into the sample and mix. A sync out pulse will be sent to the programmable pulse generator (23), which will send a series of trigger pulses to the KrF excimer laser (14) to trigger UV laser pulses. The UV laser pulse is focused by a lens (21) down onto the UV transparent capillary reaction cell (13), with a laser power meter (20) used to ensure that the laser energy per pulse is at the proper level. The frequency of pulse will be set according to the linear velocity of the syringe push, ensuring that each volume of sample is illuminated with a single laser pulse, with a buffer volume unilluminated as previously determined to be ideal for the capillary inner diameter and linear velocity to be used. The sample/photoreactive compound mix will be pulled from the sample with an air bubble before and after the sample bolus, and then injected from the needle (11) into the needle seat (12), with the switching valve in position to push the sample through the path of the laser beam in the capillary reaction cell (13) and into the sample holding loop (19). After the entire sample has been pushed through the reaction cell, the sync pulse will be turned off, stopping the laser pulses. The sample will be pulled back into the needle (11) and redeposited into its vial maintained in the heat controlled sample tray (17), to which will be added quench solution (e.g., containing catalase and methionine amide) maintained in the cooled reagent tray (18) maintained at 4° C. After incubation in the quench solution, the sample will be pulled into the needle (11) and placed into a denaturation solution (usually containing 6 M guanidinium HCl and 10 mM DTT) kept in the heat controlled denaturation tray (17) (usually at 65° C.). After denaturation, the denatured protein solution will be placed into an empty vial in the heat controlled sample tray (16) and mixed with six volumes of a dilute buffered solution of a protease (usually trypsin), maintained at 4° C. in the cooled reagent tray (18). After 3 hours of digestion at the optimum temperature for the protease (e.g., 37° C.), the sample will be pulled from the reaction vial by the needle (11) and injected into the needle seat (12), to be placed in a sample loop. After the entire sample has been loaded into the loop, the switching valve (15) will switch again, placing the loop in line with the HPLC flow and pushing the sample onto the LC column for LC-MS analysis to analyze the reacted sample. The mass spectra will also be analyzed to measure the amount of available hydroxyl radical generated in the FPOP process.

Embodiments of the present disclosure include a device that is designed to automate the process of Fast Photochemical Oxidation of Proteins (FPOP) using a UV/fluorescence detector system for measuring the amount of radical generated in the sample mixture. A diagram of the device is given in FIG. 1.2. The sample can be incubated in the heated sample tray (37) at a preset temperature (e.g., 37° C.) until ready for oxidation. The XYZ positionable syringe with pump (31) will deposit the photoactive reagent mixture (e.g., a mixture of hydrogen peroxide, glutamine radical scavenger, and AlexaFluor dosimeter reagent) into the sample and mix. A sync out pulse will be sent to the programmable pulse generator (43), which will send a series of trigger pulses to the KrF excimer laser (34) to trigger UV laser pulses. The UV laser pulse is focused by a lens (42) down onto the UV transparent capillary reaction cell (33), with a laser power meter (41) used to ensure that the laser energy per pulse is at the proper level. The frequency of pulse will be set according to the linear velocity of the syringe push, ensuring that each volume of sample is illuminated with a single laser pulse, with a buffer volume unilluminated as previously determined to be ideal for the capillary inner diameter and linear velocity to be used. The sample/photoreactive compound mix will be pulled from the sample with an air bubble before and after the sample bolus, and then injected from the needle (31) into the needle seat (32), with the switching valve in position to push the sample through the path of the laser beam in the capillary reaction cell (33) and into the sample holding loop (40). After the entire sample has been pushed through the reaction cell, the sync pulse will be turned off, stopping the laser pulses. The sample will be pulled back into the needle (31) and redeposited into its vial maintained in the heat controlled sample tray (37), to which will be added quench solution (e.g., containing catalase and methionine amide) maintained in the cooled reagent tray (39) maintained at 4° C. After incubation in the quench solution, the sample will be pulled into the needle (31) and placed into a denaturation solution (usually containing 6M guanidinium HCl and 10 mM DTT) kept in the heat controlled denaturation tray (38) (usually at 65° C.). After denaturation, the denatured protein solution will be placed into an empty vial in the heat controlled sample tray (37) and mixed with six volumes of a dilute buffered solution of a protease (usually trypsin), maintained at 4° C. in the cooled reagent tray (39). After 3 hours of digestion at the optimum temperature for the protease (e.g., 37° C.), the sample will be pulled from the reaction vial by the needle (31) and injected into the needle seat (32), with the switching valve moved to the second position, pushing the sample through the capillary reaction cell (33) (with no laser pulse) and into the loop with the UV/fluorescence detector (35). The fluorescence of the AlexaFluor dosimeter will be measured in this detector to measure the amount of available hydroxyl radical generated in the FPOP process. After the entire sample has been loaded into this loop/detector, the switching valve (37) will switch again, placing the detector/loop in line with the HPLC flow and pushing the sample onto the LC column for LC-MS analysis.

### Example 2

### Brief Introduction

Hydroxyl radical protein footprinting coupled with mass spectrometry is a powerful tool used to probe protein structure, interactions, and conformational changes in the solution phase. A hydroxyl radical dosimeter is used to ensure that different proteins oxidized under the same amount of radical. However, AlexaFluor is not an ideal dosimeter for hydroxyl radicals: it is a loss-of-signal dosimeter which lowers its sensitivity, and has several oxidation products, each with different properties. In biological research, previous reports have proposed terephthalic acid (TPA) as a hydroxyl radical dosimeter. Unlike AlexaFluor, TPA gains fluorescence upon oxidation and has only one oxidation product upon reaction with hydroxyl radicals. Our study investigated the suitability of TPA as a hydroxyl radical dosimeter for the normalization of hydroxyl radical protein footprinting data. However, further data indicates that TPA maybe not an ideal radical dosimeter to use in hydroxyl radical protein footprinting due to its complex relationship between radical dose and fluorescence. An alternative approach using tyrosine amide as both a scavenger and a radical dosimeter coupled with mass spectrometry to measure the oxidization level of tyrosine amide shows promise for normalization of footprinting data. Initial data indicates that tyrosine amide acts as a suitable replacement for glutamine as a scavenger and provides a dosimetry response that appears to be linearly related to effective hydroxyl radical dose.

### Methods

Sets of sodium phosphate buffered solutions of TPA from 1 nM to 10 mM with 100 mM hydrogen peroxide were irradiated using a 248 nm KrF excimer laser to photolyse hydrogen peroxide, forming hydroxyl radicals for oxidation. A Shimadzu LPC spectrofluorophotometer was used to obtain the excitation and emission spectra at identical wavelengths for each set of samples. Selected concentrations of TPA solution were studied, both in the presence and absence of apomyoglobin, under normal protein footprinting condition (8 μM of final protein concentration; 20 mM of glutamine concentration) and irradiated under 45 mJ/pulse laser power with 100 mM of hydrogen peroxide. A selected concentration of TPA solution was also studied by irradiating with different concentrations of hydrogen peroxide and various buffers. Catalase was then added to quench excess hydrogen peroxide, and the final products were quantitatively analyzed using the spectrofluorophotometer. The amount of oxidized product of TPA was calculated from the working curve which generated by using commercial 2-hydroxyterephthalic acid purchased from Sigma. All samples (100 μL) were diluted to 1 mL for spectrofluorophotometer analysis. In the mass spectrometry experiments, tyrosine amide (5.4 mM (Calculated concentration)) is used as a scavenger and radical dosimeter agent to replace glutamine.

### Discussion and Conclusions

FIGS. 2.1 to 2.7 illustrate data that indicating that tyrosine amide could be used as a radical dosimeter. FIG. 2.1 shows the MS spectrum FPOP of ubiquitin protein with 20 mM glutamine scavenger (top) versus FPOP of the same sample using 5.4 mM tyrosine amide as the scavenger and dosimeter. FIG. 2.2 shows the extent of ubiquitin protein oxidation in the presence of the standard 20 mM glutamine scavenger. The substitution of tyrosine amide as a scavenger/dosimeter results in an indistinguishable amount of oxidation of the ubiquitin protein (FIG. 2.4). The tyrosine amide has the added benefit of providing an internal control of the amount of available hydroxyl radical by measuring the extent of the reaction of the available radical with the tyrosine amide. This reaction is monitored by measuring the intensity of the unmodified tyrosine amide (m/z 181) versus that of the oxidized tyrosine amide (m/z 197) as shown in FIG. 2.3. As the amount of available hydroxyl radical increases, either due to a larger amount of initial radical or a lower amount of radical scavenging in solution, the oxidation of tyrosine amide varies to track that level of available radical. FIG. 2.7A shows the tyrosine amide response to differing levels of radical scavenger in solution, while FIG. 2.7B shows the tyrosine amide response to differing levels of initial hydroxyl radical produced (in this case by varying the concentration of hydrogen peroxide used during FPOP).

TPA is not an ideal radical dosimeter for protein hydroxyl radical footprinting. Glutamine, which is the scavenger normally used in protein hydroxyl radical footprinting, can interfere with the fluorescence of oxidized TPA products. The fluorescence of TPA in scavenging buffers, such as Tris, indicates that the fluorescence measured is not necessarily a function of the available radical dose. Tyrosine amide can be used, instead of glutamine, to act as a radical scavenger and also provide information as a radical dosimeter by MS detection. Initial studies indicate that the accumulation of +16 oxidation products of tyrosine amide correlate linearly with the available radical dose.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to measurement techniques and the units of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

We claim at least the following:

1. An automated device, comprising:
a sample handling system for transporting at least a sample, a photoactive reagent mixture, and a sample mixture;
a light source system for initiating a reaction in the sample mixture;
a sample flow and control system for transporting at least the sample, the photoactive reagent mixture, and the sample mixture;
a photometer system to measure light source output; and
an analysis system for analyzing the sample mixture, wherein the analysis system measures the amount of radical generated in the sample mixture.

2. The device of claim 1, wherein the analysis system includes a mass spectrometry system for measuring the amount of radical generated in the sample mixture.

3. The device of claim 1, wherein analysis system includes a LC-MS system for measuring the amount of radical generated in the sample mixture.

4. The device of claim 1, wherein the photoactive reagent mixture can include a photoactive agent, a radical scavenger, and a radical dosimeter.

5. The device of claim 1, wherein the radical scavenger is selected from the group consisting of: tyrosine amide and glutamine amide.

6. The device of claim 1, wherein the radical dosimeter includes tyrosine amide.

7. The device of claim 1, wherein the analysis system includes a UV/fluorescence detector system for measuring the amount of radical generated in the sample mixture.

8. The device of claim 1, wherein the photoactive reagent mixture can include a photoactive agent, a radical scavenger, and a UV/fluorescent agent.

9. The device of claim 1, further comprising a chamber for mixing the sample and the photoactive reagent mixture to form a sample mixture.

10. The device of claim 1, wherein the light source system is a focused pulsed light source system that includes an excimer laser.

11. The device of claim 1, wherein the photometer system includes a photometer for measuring the light output from the light source.

12. The device of claim 1, wherein analysis system includes a LC-MS system to analyze the sample after the reaction is completed.

13. An automated method, comprising:
mixing a sample with a photoactive reagent mixture to form a sample mixture;
exposing the sample mixture to light energy to initiate activation of the reaction in the sample mixture;
mixing the sample mixture with one or more other reagents;
measuring the amount of radicals in the sample mixture; and
analyzing the sample mixture.

14. The method of claim 13, wherein the steps are performed using the automated device of claim 1.

15. The method of claim 13, wherein measuring the amount of radicals in the sample mixture is performed by a mass spectrometry system.

16. The method of claim 15, wherein the mass spectrometry system is a liquid LC-MS system.

17. The method of claim 15, wherein the photoactive reagent mixture can include a photoactive agent, a radical scavenger, and a radical dosimeter.

18. The method of claim 17, wherein the radical scavenger is selected from the group consisting of: tyrosine amide and glutamine amide.

19. The method of claim 17, wherein the radical dosimeter agent includes tyrosine amide.

20. The method of claim 13, wherein measuring the amount of radicals in the sample mixture is performed by a UV/fluorescence detector system.

21. The method of claim 20, wherein the photoactive reagent mixture can include a photoactive agent, a radical scavenger, and a UV/fluorescent agent.

22. The method of claim 13, further comprising a chamber for mixing the sample and the photoactive reagent mixture to form a sample mixture.

23. The method of claim 13, wherein the light energy is a focused pulsed light from an excimer laser.

24. The method of claim 23, further comprising measuring the light energy with a photometer system includes a photometer.
